(19) United States
(12) Patent Application Publication
(10) Pub. No.: US 2026/0251627 A1
(43) Pub. Date: Aug. 27, 2026

(54) SYSTEM AND METHOD TO AUTO CALIBRATE AND AUTO BASELINE A POLLUTANT GAS DETECTOR

(71) Applicant: QUBE TECHNOLOGIES INC., Calgary (CA)

(72) Inventor: Adrian DUMITRU, Calgary (CA)

(21) Appl. No.: 19/163,372

(22) PCT Filed: Mar. 6, 2024
(86) PCT No.: PCT/CA2024/050275

§ 371 Date: Sep. 8, 2025

Related U.S. Application Data
(60) Provisional application No. 63/451,063, filed on Mar. 9, 2023.

(51) Int. Cl. G01N 33/00

(52) U.S. Cl. CPC G01N 33/0006

(57) ABSTRACT

A system and method for measuring and monitoring levels of a gas in an environment are disclosed. The system includes a sensor disposed at some distance from the source, for measuring concentration of the gas to provide gas readings; a detector module in communication with the sensor. The detector module includes a background gas detector for computing a background value for concentration of the gas; an auto-baseline and auto-calibration and auto-baseline subsystem for aligning a calibration curve for the sensor to the background value; and an external gas detector for detecting an external value for concentration of the gas, the external value different from the background value. The detector module is operable to store a calibration curve, the background value; and to cause the auto-baseline and auto-calibration subsystem to align the calibration curve to a background value based on the gas readings.

### TECHNICAL FIELD

The specification generally relates to a system and a method of auto-calibration and auto-baseline. The specification also relates to a system and method for auto-calibrating and auto-baselining a pollutant gas detection unit.

### BACKGROUND

Metal-oxide (MOx) sensors are a class of sensors that measure gas concentrations according to a single, non-linear, calibration curve applicable to all concentrations within its detectable range, including zero. As such, a shift in its output assigned to zero or background gas, called herein the baseline, can disproportionally affect its output to higher concentrations.

When metal-oxide-based gas detectors are deployed in an area of operation, sensor-specific calibration curves are individually programmed, and a calibration process is commenced after a minimum prescribed period. “Calibration process” refers to realigning the calibration curve to the amount of background gas and environmental conditions present at the site of deployment.

Depending on the operating environmental conditions, metal-oxide sensors age over time, causing a shift in their response to gas, including potentially erroneous readings of gas, including background gas. Environmental background gas refers within the scope of this disclosure to the amount of the specific gas continuously present at a specific geographic location.

Digital electrochemical (DEC) sensors are sensors that incorporate electro-chemical sensing elements that are operated under the control of microprocessors. The microprocessor in a typical digital electrochemical sensor controls the bias and power of the sensing element, acquires measurements from the sensing element, calculates gas concentration, temperature, and pressure, and provides calculated values digitally upon request from an integrating system.

A typical DEC sensor is calibrated using two gas concentrations: (i) one of zero corresponding to the sensor baseline or ‘zero’; and (ii) another higher concentration, typically half or full scale of the sensor, corresponding to the sensor ‘span’. Accuracy of sensor's gas output is computed based on the values of ‘zero’ and ‘span’, that are typically calibrated at the factory and saved in an internal memory of the microprocessor. The span calibration remains valid for an extended period in normal operation, typically more than a year. Performing a ‘span’ calibration during field operation is difficult, as only gas of a specific concentration and none of the surrounding air needs be applied to each sensor and under specific environmental conditions. Baseline needs to be performed more frequently as multiple measurement tolerances prevail at lower gas concentrations as well as possible presence of other gases at the place of operation, to which the sensor exhibits positive or negative cross sensitivity. ‘Zero’ calibration can be achieved in field operation at times when there are no pollutant gases, i.e., measured gas concentration is zero.

A calibration process should be performed regularly during gas detector deployment to realign the MOx calibration curve so that the gas detector reading matches that of environmental background gas present at its location, thus adjusting both MOx baseline and the overall gas response.

A calibration process should also be performed regularly during gas detector deployment to realign baseline of DEC present at its location that may be affected by the change in environmental conditions or the presence of gases that is cross sensitive to.

MOx calibration is initiated after a prescribed amount of time following the gas detector field deployment and after calibration curves have been programmed. Manually managing these durations of time for a large number of detectors is challenging.

MOx calibration should only be initiated when there is no gas with which it reacts. Otherwise, the calibration curve will be misaligned and causing detector's gas response to become erroneous for the entire range.

DEC baseline should only be initiated when there is no gas with which it reacts. Otherwise, it may respond inaccurately at lower gas concentrations or respond to no gas with negative gas concentrations, a physical impossibility.

Manual calibrating and baselining processes for either MOx or DEC have a range of disadvantages. They are often error prone due to human error, often demand a large investment in time and labor and if improperly done can result in improper pollutant gas response.

Calibration and baseline processes must be initiated when there is a shift in sensor response due to aging that may be difficult for an operator to gauge without constant monitoring and dedicated experience, i.e., determining whether higher gas reading is due to sensor aging or higher amounts of gas present.

In order to minimize cellular data transmission charges, gas detectors often send to a remote operator only those readings that are significantly different from the previous ones. Without access to all gas readings and analysis of the data, any slow trends in the background gas reading may be difficult to detect by the operator as soon as they happen.

In addition, managing calibration and baseline schedules of many gas detectors can consume excessive amounts of time from the remote operator. Discerning a rise in detector's response between sensor ageing and true gas response requires extended and dedicated observation from the remote operator.

Accordingly, improved systems and methods of calibration and baseline are desired. Embodiments presented in the present disclosure aim to overcome at least some of the aforementioned problems.

### SUMMARY OF THE DISCLOSURE

In accordance with one aspect of the present disclosure, there is provided a system for measuring and monitoring a gas in an environment, the system comprising: (i) a gas sensor disposed a distance from a source emitting the gas, for measuring concentration of the gas and for providing gas readings; and (ii) a detector module in communication with the gas sensor, the detector module comprising a processor and memory storing processor executable instructions that, when executed, implement: (a) a background gas detector for computing a background value for the concentration of the gas in the presence of external gas; (b) an auto-baseline and auto-calibration subsystem for aligning a gas sensor calibration curve for the gas sensor to the background value; and (c) an external gas detector for detecting an external value for the concentration of the gas, the external value being different from the background value. The detector module is operable: (i) to store the gas sensor calibration curve, and an environmental value for the gas; and (ii) to cause the auto-baseline and auto calibration subsystem to align the gas sensor calibration curve to the background value.

The system may further comprise a messaging subsystem. The messaging subsystem may be operable to exchange one or more messages with remote operator of the system. One or more messages may comprise one or more of: (i) an indication that the gas sensor does not require calibration or baseline; (ii) an indication that the sensor requires calibration or baseline; and (iii) an indication that the auto-baseline and auto calibration subsystem has been triggered and that the detector module is waiting to issue a calibration or a baseline command. Auto-calibration and auto-baseline of the system can be enabled or disabled in response to one or more commands received from the remote operator.

The gas sensor may comprise a metal oxide semiconductor (MOx) sensor and one or more digital electro-chemical (DEC) sensors.

The background gas detector may automatically initiate a gas sensor calibration or a gas sensor baseline process upon determining that the background value is different from the environmental value by more than the first threshold. The background gas detector may compute the background value as a first running average B_{N} of the gas readings excluding the gas readings that are higher than the first running average. The external gas detector may compute the external value as a second running average E_{N} of all of the gas readings.

The detector module may adjust the gas sensor calibration curve to account for changes in ageing or exposure to degrading elements.

In accordance with one aspect of the present disclosure, there is provided a method for measuring and monitoring a gas in an environment, the method including: using a gas sensor disposed a predetermined distance from a source emitting the gas, measuring a concentration of the gas and providing one or more gas readings; using a background gas detector, computing a background value for the concentration of the gas in the presence of external gas; using an auto-baseline and auto-calibration subsystem, aligning a gas sensor calibration curve for the gas sensor to the background value; using an external gas detector, detecting an external value for the concentration of the gas, the external value being different from the background value; storing the gas sensor calibration curve, and an environmental value; and using the auto-baseline and auto calibration subsystem, aligning the gas sensor calibration curve to the background value upon determining that the background value is different from the environmental value by more than the first threshold.

The background value at an N^{th} gas reading, denoted B_{N}, may be computed based on the previous background value at the (N-1)^{st} gas reading, denoted B_{N-1}, and the current gas reading, denoted G_{N}, according to the formula:

$${B}_{N} = \frac{{B}_{N - 1} ( N - 1 ) + {G}_{N}}{N} .$$

G_{N} may exclude readings from: (a) a prescribed time period associated with detector power up; (b) a prescribed time period after the gas sensor calibration curve has been stored; and (c) external gas readings.

The method may further comprise: upon determining that B_{N} differs from a programmed background value by a predetermined threshold, sending a message indicative of said determining to a remote operator of the auto-baseline and auto-calibration subsystem.

Other technical advantages may become readily apparent to one of ordinary skill in the art after review of the following figures and description.

### BRIEF DESCRIPTIONS OF THE DRAWINGS

For a better understanding of the embodiments described herein and to show more clearly how the embodiments may be carried into effect, reference will now be made, by way of example only, to the accompanying drawings in which:

FIG. 1 a schematic block diagram of several hardware components of a gas detection and monitoring system according to one example of an embodiment of the present disclosure;

FIG. 2 is a simplified schematic block diagram of software modules executing within the gas detection and monitoring system of FIG. 1;

FIG. 3 is a flowchart of a method carried out by the background detector software module of FIG. 2; and

FIG. 4 is a flowchart of a method carried out by the auto-baseline and auto-calibration software module of FIG. 2.

### DETAILED DESCRIPTION

Described herein is a gas monitoring and detection system comprising one or more detector modules wherein each detector module can continuously monitor concentrations of a desired gas while at same time discerning the background and the external gas component of that concentration. Background component is compared to the environmental background value of the desired gas and when the difference exceeds a predetermined threshold value, the system auto-generates a calibration command in the case of MOx sensors and a baseline command in the case of DEC sensors. Environmental background value of a specific gas refers to the concentration present at the specific location when no pollutant gas is present. A “Calibration command” adjusts the sensors' response to all concentration levels in its range, including zero level, whereas a “Baseline command” adjusts the response to concentrations within the lower half of its range.

Such a system improves gas reading accuracy and gas detector fleet efficiency by automatically detecting shifts in sensors' calibration and baseline as soon as they happen and auto triggering auto-calibration and auto-baseline without the need for any manual intervention.

Directional terms such as “top,” “bottom,” “upwards,” “downwards,” “vertically,” and “laterally” are used in the following description for the purpose of providing relative reference only, and are not intended to suggest any limitations on how any article is to be positioned during use, or to be mounted in an assembly or relative to an environment. The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

In this disclosure, “baseline process” refers to realigning sensor response to gas concentrations in the lower half of its range and other relevant environmental conditions present at the site of deployment of the gas detector system.

In this disclosure, “calibration process” refers to realigning sensor calibration curve to the concentration of environmental background gas and other relevant environmental conditions present at the site of deployment of the gas detector system.

In this disclosure, the terms “comprising”, “having”, “including”, and “containing”, and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method, or use functions. The term “consisting of” when used herein in connection with a composition, use, or method, excludes the presence of additional elements and/or method steps.

In this disclosure, “external gas” refers to gas that is emitted from a source and that exceeds the environmental background concentration level of such gas at the location of a gas detector system.

In this disclosure, “gas” refers to any gas pollutant to the environment.

In this disclosure, “gas reading” or “gas sample” refers to a gas value that has already taken into account relevant environment variables such as, but not limited to, temperature, pressure, and humidity.

In this disclosure, “remote operator” refers to a software system that one or more gas detectors are controlled by and send data to and that is operated by an operator who is not located at the site of deployment of the gas detector system.

### System Overview

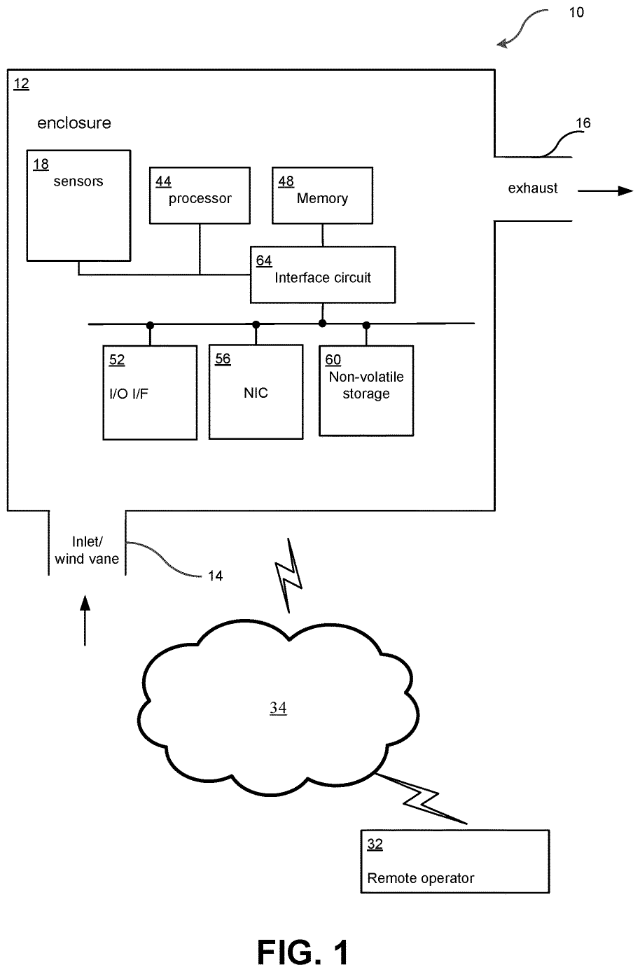

FIG. 1 depicts a simplified block diagram of a gas detection and monitoring system 10 which is an example of an embodiment of a gas detection and monitoring system described in the present disclosure. As shown in FIG. 1, system 10 includes an enclosure 12 that houses a sensor 18 and other circuitry and hardware components which will be described below. Enclosure 12 has an inlet 14 for receiving air that may contain various concentrations of one or more gases of interest, and an exhaust 16 for expelling air out of the enclosure 12. In other embodiments, there may be two or more gas sensors 18.

A remote operator 32 exchanges command, control, status, and other data with the detector module 20 via a network 34 using wired, wireless or a combination of both wired and wireless means. Remote operator 32 refers to a software-based system that the system 10 sends data to and receives control commands from, and which may be fully or partially operated by humans or fully automated.

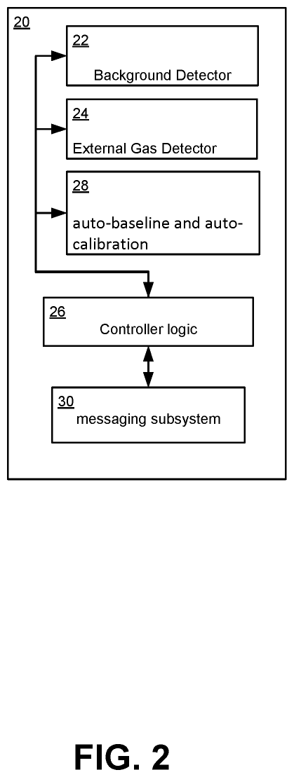

In the depicted embodiment, system 10 includes a number of physical components including a processor 44, which may be in the form of a central processing unit (“CPU”), a microprocessor, or a microcontroller, as well as memory in the form of a volatile random-access memory (“RAM”) 48 and non-volatile storage 60. System 10 includes an input/output (“I/O”) interface 52, a network interface 56, and an interface circuit 64 which enables processor 44 to communicate with the other components. Processor 44 executes processor executable instructions in the form of at least an operating system, and one or more applications including the software modules depicted in FIG. 2.

RAM 48 provides relatively responsive volatile storage to processor 44. I/O interface 52 allows input to be received from one or more external components such as sensor 18 and outputs information to output devices, such as a display or fan. Network interface 56 permits wired or wireless communication with other computing devices over computer networks such as the Internet. Non-volatile storage 60 stores the operating system and programs, including computer-executable instructions for implementing the software implemented portions of software modules in the form of background detector 22, external gas detector 24, and auto-baseline and auto-calibration subsystem 28 and associated code, data structures and objects. During operation of detector module 20 in the gas detection and monitoring system 10, operating system, the programs and the data may be retrieved from non-volatile storage 60 and placed in RAM 48 to facilitate execution.

In this embodiment, sensor 18 is a metal oxide semiconductor (MOx) and one or more digital electro-chemical sensors (DEC). In other embodiments, two or more sensors which may or may not be MOx or DEC types can be used.

An auto-baseline and auto-calibration feature, which will be discussed later, may be used with other sensor technologies.

According to this embodiment, sensor 18 includes one or more gas sensors and other environmental sensors and associated controllers for detecting one or more of gas concentration, temperature, humidity, pressure, or other values. The detected values are read by detector module 20. Detector module 20 reads all of the values and compensates for environmental variables such as, but not limited to, temperature, humidity, and pressure, as needed in another software module.

FIG. 2 is a simplified schematic block diagram of software modules and subsystems executing within the gas detection and monitoring system of FIG. 1. In the depicted embodiment, detector module 20 is executed by processing hardware which includes processor 44 interacting with memory 48 and storage 60 storing processor executable instructions in the form of software or firmware which will be described later. Detector module 20 is operable to receive data, including gas concentration readings obtained from sensors 18 and to send control data to sensors 18.

As depicted, detector module 20 implements a plurality of subsystems or software modules including background detector 22, external gas detector 24, auto-baseline and auto-calibration subsystem 28 and a messaging software module 30. A controller logic 26 interacts with the various subsystems or modules.

Messaging software module 30 is a software module that communicates with remote operator 32 to provide information regarding gas sensor calibration status. Messaging software module 30 may utilize a communication submodule such as network interface 56 adapted to provide cellular, satellite, or Wi-Fi™ data transmission, global positioning satellite (GPS), Bluetooth™ and other interfaces. Messaging software module 30 in this embodiment is hardware interface agnostic capable of utilizing cellular and Wi-Fi™ interfaces, and is operable to send one or more messages that include information such as an indication that the sensors 18 do not require calibration, that the sensors 18 require calibration, that the auto-calibration or auto-baseline process has been triggered and that the detector is waiting for a “no gas moment” to issue the and auto-calibration or auto-baseline command.

A “No gas moment” is defined as a time when there has not been any external gas detected in a previous pre-defined period, usually in the magnitude of minutes. In practical terms that means that outputs of the sensor 18 has been relatively constant before a ‘No gas moment’. External gas brought in by gusting wind into enclosure 12 causes spikes in the output of the sensor 18.

MOx sensors used in sensor 18 typically have one compensation curve over the entire gas range. As the sensor 18 becomes exposed to the elements, this curve often shifts but it takes only one gas concentration to pin back. The gas concentration used in this embodiment is the environmental background gas concentration, which is a known quantity at any geographic location, as noted above.

This is the main difference between other sensor technologies including DEC discussed above that require two concentrations—at 0 and at top of the range to pin back their curve. These concentrations are nearly impossible to apply to sensors in the field.

### Background Detector

According to an embodiment of the system, background detector 22 is at least partly implemented in software via processor executable instructions executing on a processor such as processor 44, to calculate the background concentration of a gas or background value, during times that an external gas is either not present or temporarily present at the location of the sensor 18. In the depicted embodiment, background detector 22 is an example of a software system.

A gas detection and monitoring system such as system 10 may have up to six sensors, one or more of which may be of the MOx type and one or more of the DEC type. As noted above auto-calibration is generally applicable to a MOx type sensor and the auto-baseline is applicable to DEC type sensor.

As noted above, auto-calibration for the MOx type sensor results adjusts its response to all gas concentrations including zero and top of the range. Auto-baseline for the DEC sensor adjusts its response to gas concentrations to the lower half of its range including 0.

Sensor 18 is typically placed at a distance of 25 metres or more from one or more potential gas sources. Typically, wind directs external gas from its source to a gas detection and monitoring system such as system 10. In most cases, sources of external gas are uneven. In addition, the velocity and the direction of the wind that guides the gas from its source to the detector are also uneven.

Consequently, external gas may be found to be present at the location of sensor 18 in very uneven or non-uniform concentrations or durations.

A plot of concentration of external gas (on the vertical axis) against time (on the horizontal axis), in the vicinity of the detector may show a temporary succession of short peaks or crusts of varying magnitudes, followed by valleys or troughs, some of which are close to the background levels for the area. Gas readings are taken continuously along with readings of other sensors or gases. Each reading takes time to acquire and process. The MOx and DEC sensor 18 in the depicted embodiment is sampled at intervals of 6-7 seconds. All the samples are available to the auto-baseline and auto-calibration algorithm but only a small subset of the samples are sent to remote operator 32.

According to another embodiment of the system 10, background detector 22 can maintain a running average of background gas readings obtained from sensor 18. These background gas readings exclude samples from: (a) a prescribed time period associated with detector power up; (b) a prescribed time period after the calibration curve have been programmed in the detector module; and (c) external gas readings. Gas readings that are not excluded under the above conditions are collectively called “included samples”.

Gas readings are considered “external gas readings” when they are deemed significantly higher (e.g., by at least the sensor resolution for MOx and DEC senor, or a pre-set percentage (e.g., 20%) of a programmed background value for the MOx sensor, or a combination of the two) than the current computed background level. Successive external gas reading samples may be excluded from the running average or cumulative average up to a pre-defined maximum number called “Maximum Peak Exclusion Samples”. A counter called herein ‘Peak Exclusion Samples’ will count the number of consecutive samples excluded from the computed running average.

If, following a series of external gas readings during the period of Maximum Peak Exclusion Samples, a gas reading comes close to the current background calculated value, then the sample is once again added to the background average (thus becoming an included sample) and the number from ‘Peak Exclusion Samples’ is reset to 0.

In one embodiment, “close” means within the resolution of the sensor 18. As an example, if the resolution of a MOx type sensor 18 is 1 ppm (parts per million) and calculated background value is 3 ppm, then readings over 3 ppm+1 ppm (i.e., >4 PPM) are considered external gas and those that are less are considered background gas. Background detector 22 allows for a maximum number of consecutive samples (called Maximum Peak Exclusion Samples) to be excluded from the running average. A counter or timer that counts towards that maximum is called Peak Exclusion Samples.

If the number of external gas readings reaches the Peak Exclusion Time, then further higher gas readings will be added to the running average. Such implementation is based on the fact that gas ‘peaks’ are expected to be short and last a few to several readings followed by ‘valleys’ that are close to the gas background value.

A background value at the N^{th} gas reading, denoted B_{N}, is computed based on the previous background value at the (N-1)^{st} gas reading, denoted B_{N-1}, and the current gas reading, denoted G_{N}, according to the formula in equation (1) below:

$${B}_{N} = \frac{{B}_{N - 1} ( N - 1 ) + {G}_{N}}{N} \qquad ( 1 )$$

The computation of the above background value in equation (1) may be seen as a cumulative average computation of all included samples of gas readings since the last calibration or baseline. The above formula in equation (1) is more efficient than a brute force method that simply stores and sums all of the gas readings G₁, G₂, . . . . G_{N} and then divide the sum by the total number of readings N as shown in equation (3).

However, if the previous cumulative average B_{N-1} is known, it is possible to more efficiently calculate the new cumulative average B_{N} based on the previous cumulative average B_{N-1} as each new gas reading G_{N} becomes available, as derived below.

The previous cumulative average B_{N-1} is computed as:

$${B}_{N} = \frac{{G}_{1} + {G}_{2} + … + {G}_{N - 1}}{N - 1} \qquad ( 2 )$$

After the N^{th} gas reading G_{N}, the background value B_{N} becomes:

$${B}_{N} = \frac{{G}_{1} + {G}_{2} + … + {G}_{N - 1} + {G}_{N}}{N} \qquad ( 3 )$$

Since the sum of previous N-1 readings G₁+G₂+ . . . +G_{N}-1=B_{N-1} (N-1) according to equation (2), it is possible to substitute B_{N-1} (N-1) for the sum of previous N-1 gas readings or samples in equation (3), which simplifies the result as shown in equation (1) above.

As may be appreciated, each of the gas readings G₁, G₂, . . . , G_{N} that is used to compute the above cumulative average or background value in equation (4) is among the included samples.

Intelligent selection of samples, as described herein, for inclusion or exclusion in the computation the cumulative value to obtain a background value leads to improved gas detection.

### Auto-Calibration and Auto-Baseline

Auto-baseline and auto-calibration subsystem 28 is used to re-establish a sensor calibration and baseline as noted above, after anomalous readings are obtained. If, after a prescribed period ranging from two hours to several days, auto-baseline and auto-calibration subsystem 28 receives a calculated background value that is significantly different than the environmental background value programmed in the gas detector, then an auto-calibration or auto-baseline command is issued for sensor 18, depending on if the sensor is of MOx or DEC respectively as explained above. Otherwise, system 10 invokes the background detector 22 for the subsequent time frame.

In the case of methane detection for example, a known global environmental average background is around 1.8 ppm. In practice, the specific environmental value varies based on the particular geographic location on earth and may range from about 1.6 ppm to about 2.15 ppm. These predetermined environmental values are available in geographic datasets that provide the methane background value for any specific location. Environmental methane background value is greater than the 1 ppm resolution of the MOx type sensor used to measure it within the scope of this disclosure and is programmed gas detector storage 60.

In the case of hydrogen sulphide (H₂S) detection for example, a known global environmental average background value is less than about 1 ppb. Environmental H₂S background value is smaller than the resolution of the DEC type sensor used to measure it within the scope of this disclosure, and it is the value of 0 programmed in gas detector storage 60.

When the physical gas detection and monitoring system 10 is installed, the environmental gas background value for the location is programmed into the system (e.g., in storage 60). The background detector 22 calculates the background value B_{N} as described above and compares it to the environmental value that had been programmed in the gas detector. If the two values are found to be close to each other, then no action is taken other than informing the remote operator 32 that the sensor 18 is in calibration or in baseline. If the calculated value B_{N} from background detector 22 is significantly different (e.g., by at least the resolution, or a pre-set percentage (e.g., 20%) of a programmed background value for the MOx sensor, or a combination of both) than the programmed environmental value, then detector module 20 informs the remote operator 32 of this fact; if the auto-baseline and auto-calibration is enabled, detector module 20 self-generates an auto-baseline or an auto-calibration command. Gas samples G_{N} are obtained every 6-7 seconds. A typical cycle to evaluate the background is in the range varying from one hour to multiples of 24 hours depending on the sensor and environmental conditions.

### External Gas Detector

Before an actual auto-calibration or auto-baseline command is auto-generated, the auto-baseline and auto-baseline and auto-calibration subsystem 28 confirms that there is no external gas detected. As contemplated in system 10, external gas detector 24 is utilized. The output of the same sensor 18 is used for both background detector 22 and external gas detector 24, both of which are software implemented systems.

Both background detector 22 and external gas detector 24 compute running average but the main difference between background detector 22 and external gas detector 24 is that the background detector 22 excludes external gas samples (i.e., samples that are higher than its computed average) whereas the external gas detector 24 includes them. The purpose of background detector 22 is to compute the background gas only and of the latter to compute all the gas, so to ensure that there is no external gas when auto-calibration or auto-baseline command is issued.

Another difference between background detector 22 and external gas detector 24 relates to time frames involved. Background detector 22 has a time frame usually more than an hour whereas the external gas detector 24 has a time frame of minutes. The latter has the purpose of ensuring that there has not been any gas present in the few minutes prior to auto-baseline. As external gas presents itself to the gas detector in groups of spikes close to each other as noted above, there is a likelihood that there will be no external gas when the auto-baseline or auto-calibrations commands have been triggered by auto-baseline and auto-calibration subsystem 28, if there has not been any external gas in the prior few minutes.

External gas detector 24 similarly computes a running average or a cumulative average of all gas readings that do not fall under the exclusion conditions (a) and (b) identified above, for a prescribed period of time which may range from several minutes to an hour. That is, gas readings within time period t_{POWER-UP} following a detector power up; or gas readings within a time window t_{CALIBRATION} following a calibration curve being programmed in the detector module 20 will be excluded from the computation by external gas detector 24.

Sensor 18 provides raw output samples that are then computed in software using a digital filter e.g., an infinite impulse response (IIR) filter and the output of the IIR filter is fed to the software modules shown in FIG. 2. Programming a calibration curve does not affect the output of a sensor, but may change the coefficients of the IIR filter that resulting in unstable output for a period of time which is a feature of these IIR filters and thus software modules (e.g., external gas detector 24), should wait out this period.

As discussed above in general for cumulative averages, an external gas detector value denoted E_{N}, at the N^{th} gas reading G_{N} may be computed based on the previous external gas detector value E_{N-1} at the (N-1)^{st} gas reading, according to the formula:

$${E}_{N} = \frac{{E}_{N - 1} ( N - 1 ) + {G}_{N}}{N} \qquad ( 4 )$$

External gas detector 24 continuously computes the external gas value E_{N} during operation of the background detector 22 and be ready to evaluate as soon as auto-baseline and auto-baseline and auto-calibration subsystem 28 is triggered. External gas is considered not to be present when the gas detector value is close to or equal to the background detector value. If external gas is present, then the values of external gas detector 24 and background detector 22 are computed continuously until they become equal or close to each other.

In some embodiments, the gas sensor auto-calibration and auto-baseline process may be remotely enabled or disabled. When auto-baseline and auto-baseline and auto-calibration subsystem 28 has been triggered and auto-calibration or auto-baseline is enabled, if there is no external gas then a calibration or a baseline command is auto generated by the gas detector. When the auto-calibration or auto-baseline is disabled, the detector computing software module 20 sends a command to the remote operator 32 that system 10 requires calibration or baseline. Following calibration or baseline command auto generation or manual calibration or baseline, system 10 commences background computation and calibration or baseline of the following time frame.

Messages from the messaging software module 30 include information such as: an indication that the sensor 18 does not require calibration or baseline, an indication that the sensor 18 requires calibration or baseline, an indication that the auto-baseline and auto-calibration process has been triggered, and an indication that the detector computation software module 20 is waiting for a “no gas” moment to issue the calibration or baseline command.

In operation, background detector 22 and external gas detector 24 are operated to measure the amount of gas present as indicated by sensor 18 and based on the data, trigger baseline using the auto-baseline and auto-baseline and auto-calibration subsystem 28 or via commands received via messaging software module 30.

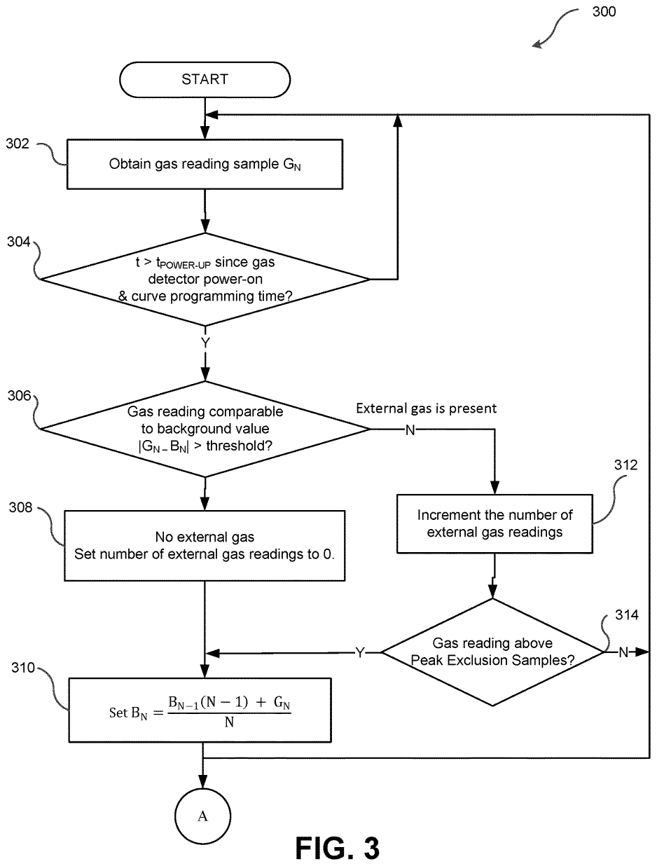

FIG. 3 depicts a flowchart 300 of a method carried out by the background detector 22. As shown, the method starts by obtaining a gas reading sample G_{N} at step 302. At step 304 the method checks if enough time has elapsed since power up of the detector software module and/or the current curve has been programmed (i.e., if t>t_{POWER-UP} since gas detector power-on and curve programming time).

At step 306, the gas reading is compared to the background value (i.e., |G_{N}−B_{N}|>threshold). If the gas reading is comparable to the background value, then the method proceeds to step 308 and the number of external gas readings is set to 0. If the gas reading is not comparable to the background value, then an external gas is deemed to be present and the method proceeds to step 312 to increment the number of external gas readings.

At step 314, if the gas reading is not above the peak exclusion samples, then the method proceeds to the starting step 302. Otherwise, the method proceeds to step 310 and the cumulative background gas level, as discussed in equation (1) or equation (4), is computed.

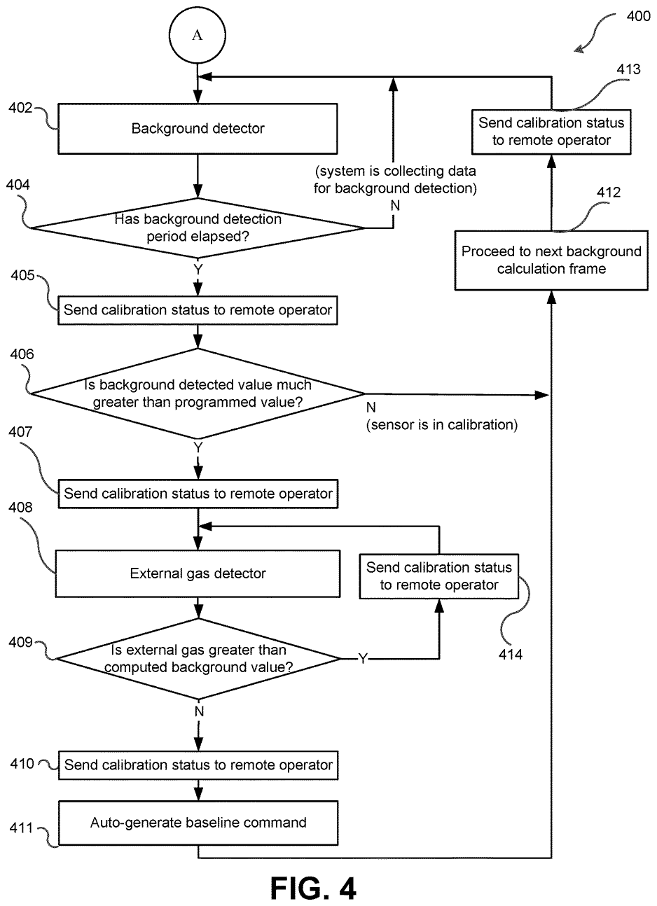

FIG. 4 depicts a flowchart of a method 400 carried out by the auto-baseline and auto calibration software module 22 of FIG. 1.

At step 402 a background detection step is executed.

At step 404 the method checks if the background detection period has elapsed. If the background detection period has not elapsed, then the system 10 is deemed to be collecting data for background detection and the method returns to step 402. Otherwise, at step 405 the method sends calibration or baseline status to the remote operator 32.

At step 406, the background detected value is checked to determine if such value is significantly greater than the programmed value (e.g., by at least the resolution, or a pre-set percentage (e.g., 20%) of the programmed value, or combination of both). If the background detected value is not greater than the programmed value, then the sensor has proper calibration or baseline, and the method proceeds to step 412. At step 413 a calibration or baseline status is sent to the remote operator 32; thereafter, the auto-baseline and auto-calibration software resets to step 402.

If the background detected value is significantly different than the programmed value, indicating that a calibration or a baseline is needed, the method proceeds to step 408 to execute external gas detector 24.

At step 409, the external gas value is compared to the computed background value. If the external gas value is not significantly greater than the computed background value, then a calibration or baseline status is sent to the remote operator 32 (see step 410), then the auto-baseline or auto-calibration commands are sent to the respective sensor (step 411), and then the cycle resets to step 402. If the external gas value is greater than the computed background value, then a calibration or baseline status is sent to the remote operator 32 (see step 414), and thereafter the auto-baseline and auto-calibration and-auto-baseline software resets to step 408, continuing the cycle until the external gas dissipates.

At step 411 a calibration or a baseline command is generated. The method then proceeds to step 412 to proceed to the next background calculation frame; thereafter, a calibration or baseline status is sent to the remote operator 32 at step 413 and the auto-baseline and auto-calibration software resets to step 402. Having completed this operation, another cycle of background calculation is initiated.

The system described herein has the ability to conduct auto-baseline and auto-calibration, thereby reducing the need to deploy operators to remote sites to perform manual calibration or manual baseline. Such system reduces the time and cost associated with fixing the effects of inaccurate readings that may otherwise be obtained.

Further, auto-baseline and auto-calibration procedures ensure that the most accurate and up-to-date data is being obtained and delivered to customers and other end users.

Although specific advantages have been enumerated above, various embodiments may include some, none, or all of the enumerated advantages.

Persons skilled in the art will appreciate that there are yet more alternative implementations and modifications possible, and that the above calibration and baseline examples are only illustrations of one or more implementations. The scope, therefore, is only to be limited by the claims appended hereto and any amendments made thereto.

What is claimed is:

1. A system for measuring and monitoring a gas in an environment, the system comprising:
a gas sensor disposed a distance from a source emitting the gas, for measuring concentration of the gas and for providing gas readings;
a detector module in communication with the gas sensor, the detector module comprising a processor and memory storing processor executable instructions that, when executed, implement:
a background gas detector for computing a background value for the concentration of the gas in the presence of external gas;
an auto-baseline and auto-calibration subsystem for aligning a gas sensor calibration curve for the gas sensor to the background value;
an external gas detector for detecting an external value for the concentration of the gas, the external value being different from the background value;
wherein the detector module is operable: (i) to store the gas sensor calibration curve, and an environmental value for the gas; and (ii) to cause the auto-baseline and auto calibration subsystem to align the gas sensor calibration curve to the background value.

2. The system of claim 1, wherein the gas sensor comprises a metal oxide semiconductor (MOx) sensor and one or more digital electro-chemical (DEC) sensors.

3. The system of claim 1 further comprising a messaging subsystem.

4. The system of claim 3, wherein the messaging subsystem is operable to exchange one or more messages with remote operator of the system.

5. The system of claim 4, wherein the one or more messages includes one or more of: an indication that the gas sensor does not require calibration or baseline; an indication that the sensor requires calibration or baseline; and an indication that the auto-baseline and auto calibration subsystem has been triggered and that the detector module is waiting to issue a calibration or a baseline command.

6. The system of claim 4, wherein auto-calibration and auto-baseline of the system can be enabled or disabled in response to one or more commands received from the remote operator.

7. The method of claim 6, wherein the one or more commands are received via the messaging subsystem.

8. The system of claim 6, wherein the background gas detector sends a message to the remote operator regarding whether the gas sensor requires calibration or baseline.

9. The system of claim 8 wherein the message comprises information indicating a need perform a manual sensor calibration or sensor baseline.

10. The system of claim 3, wherein the environmental value can be remotely programmed into the detector module via the messaging subsystem.

11. The system of claim 1, wherein the background gas detector automatically initiates a gas sensor calibration or a gas sensor baseline process upon determining that the background value is different from the environmental value by more than the first threshold.

12. The system of claim 1, wherein the background gas detector computes the background value as a first running average B_{N} of the gas readings excluding the gas readings that are higher than the first running average.

13. The system of claim 12, wherein the external gas detector computes the external value as a second running average E_{N} of all of the gas readings.

14. The system of claim 1, wherein the detector module adjusts the gas sensor calibration curve to account for changes in ageing or exposure to degrading elements.

15. A method for measuring and monitoring a gas in an environment, the method comprising:
using a gas sensor disposed a predetermined distance from a source emitting the gas, measuring a concentration of the gas and providing one or more gas readings;
using a background gas detector, computing a background value for the concentration of the gas in the presence of external gas;
using an auto-baseline and auto-calibration subsystem, aligning a gas sensor calibration curve for the gas sensor to the background value;
using an external gas detector, detecting an external value for the concentration of the gas, the external value being different from the background value;
storing the gas sensor calibration curve, and an environmental value; and
using the auto-baseline and auto calibration subsystem, aligning the gas sensor calibration curve to the background value upon determining that the background value is different from the environmental value by more than the first threshold.

16. The method of claim 15, wherein the predetermined distance is about 25 m.

17. The method of claim 15, wherein the background value at an N^{th} gas reading, denoted B_{N}, is computed based on the previous background value at the (N-1)^{st} gas reading, denoted B_{N-1}, and the current gas reading, denoted G_{N}, according to the formula:
$${B}_{N} = \frac{{B}_{N - 1} ( N - 1 ) + {G}_{N}}{N} .$$

18. The method of claim 17, wherein G_{N} excludes readings from:
(a) a prescribed time period associated with detector power up;
(b) a prescribed time period after the gas sensor calibration curve has been stored; and
(c) external gas readings.

19. The method of claim 17, further comprising: upon determining that B_{N} differs from a programmed background value by a predetermined threshold, sending a message indicative of said determining to a remote operator of the auto-baseline and auto-calibration subsystem.
